(19) United States
(12) Patent Application Publication
(10) Pub. No.: US 2024/0427950 A1
(43) Pub. Date: Dec. 26, 2024

(54) METHOD AND APPARATUS FOR WEIGHT REDUCTION OF AUTOMOTIVE BODY

(71) Applicant: JFE Steel Corporation, Tokyo (JP)

(72) Inventor: Takanobu Saito, Tokyo (JP)

(21) Appl. No.: 18/274,018

(22) PCT Filed: Oct. 1, 2021
(86) PCT No.: PCT/JP2021/036448

§ 371 Date: Jul. 25, 2023

(30) Foreign Application Priority Data
Jan. 27, 2021 (JP) 2021-010846

(51) Int. Cl. G06F 30/15

(52) U.S. Cl. CPC G06F 30/15

(57) ABSTRACT

An automotive body weight reduction method includes: acquiring an automotive body model including automotive parts modeled by a plurality of elements and joining points; obtaining sensitivity of each element; determining a dividing position of the automotive parts and/or the automotive parts to be integrated based on the sensitivity of each element; dividing and/or integrating the automotive parts, and generating an optimization analysis model; setting an objective regarding a body mass of the optimization analysis model and a constraint regarding automotive body performance of the optimization analysis model, and setting a load and constraint condition given to the optimization analysis model; and performing the optimization analysis of the sheet thickness under the load and constraint condition and the optimization analysis condition set in the sheet thickness optimization analysis condition setting step, and obtaining an optimized sheet thickness of each of the automotive parts in the optimization analysis model.

### TECHNICAL FIELD

This disclosure relates to a method and apparatus for weight reduction of an automotive body, and more particularly to a method and apparatus for weight reduction of an automotive body capable of efficiently and sufficiently reducing the weight of the automotive body while maintaining automotive body characteristics by changing a dividing position of automotive parts for the automotive body the dividing position of which into the automotive parts is fixed in advance, the automotive body including a plurality of automotive parts such as of automobiles.

### BACKGROUND

In recent years, weight reduction of an automotive body due to environmental problems has been promoted particularly in the automobile industry, and computer aided engineering (CAE) analysis has become an essential technique for automotive body design. In such CAE analysis, stiffness analysis, crashworthiness analysis, vibration analysis and the like are performed, which greatly contribute to weight reduction of the automotive body and improvement of automotive body performance.

In addition, in CAE analysis, it is known that not only simple performance evaluation, but also optimization techniques such as mathematical optimization, dimension optimization, shape optimization, and topology optimization can be used to improve various automotive body performances and reduce the weight of the automotive body. As such an optimization technique, for example, JP 2010-250818 A discloses a method for topology optimization of components of a complex structural body.

Further, JP 2020-60820 A discloses a method of performing sensitivity analysis of automotive parts with respect to automotive body performance using an optimization technique, and clarifying the automotive parts to be subjected to measures for weight reduction of the automotive body and improvement of the automotive body performance based on a result of the sensitivity analysis.

The method disclosed in JP '820 models an automotive part for an automotive body a dividing position of which into the automotive parts is fixed in advance, calculates sensitivity of each element used in the model with respect to automotive body performance by sensitivity analysis, obtains sensitivity for each automotive part based on the calculated sensitivity of each element, and clarifies an automotive part to be subjected to measures such as changing of a sheet thickness and a material property.

In that method, even when there is a sensitivity distribution in the same automotive part, the magnitude of the sensitivity is determined for each automotive part, and thus, the sheet thickness and the material property of the automotive part determined to be subjected to measures are changed. Therefore, even for an automotive part the sheet thickness or the like of which has been determined to be changed, there may be a portion of the automotive part where the sheet thickness or the like should not be changed, and since the dividing position is fixed, the automotive body performance may not be efficiently and sufficiently improved even when the sheet thickness or the like of the automotive part is changed.

Hence, when the automotive body is divided into a plurality of automotive parts, or the plurality of automotive parts is integrated, and the sheet thickness or the material property is appropriately set for each new automotive part that has been divided or integrated again, it is considered that the weight reduction of the automotive body can be efficiently and sufficiently achieved while maintaining the automotive body performance.

As a method of determining division or integration of automotive parts, a method performed based on stress or strain generated by a load applied to the automotive part is possible. In that method, it is possible to determine a boundary between a portion having a large stress or the like and a portion having a small stress or the like in the automotive part as the dividing position, and determine to integrate the automotive parts having the same degree of stress or the like.

However, in that method, it is completely unclear whether the automotive body can be reduced in weight while maintaining the automotive body performance even when the automotive parts are divided or integrated to increase the sheet thickness of the automotive part having a large stress or the like and reduce the sheet thickness of the automotive part having a small stress or the like.

It could therefore be helpful to provide a method and apparatus for weight reduction of an automotive body capable of efficiently and sufficiently reducing the weight of the automotive body while maintaining the performance of the automotive-body.

### SUMMARY

I thus provide:

An automotive body weight reduction method reduces a weight of an automotive body model including a plurality of automotive parts, is executed by a computer and includes: an automotive body model acquisition step of acquiring the automotive body model including the plurality of automotive parts modeled by a plurality of elements and joining points for joining the plurality of automotive parts as a parts assembly: a sensitivity analysis step of setting an objective regarding automotive body performance of the automotive body model, a constraint regarding a volume of the automotive body model, and a load and constraint condition or only a loading condition given to the automotive body model, and obtaining sensitivity of each element satisfying the objective under the load and constraint condition or only the loading condition and the constraint: an automotive part dividing position/integration determination step of determining a dividing position of the automotive parts and/or the automotive parts to be integrated based on the sensitivity of each element: a sheet thickness optimization analysis model generation step of dividing and/or integrating the automotive parts for which the dividing position and/or integration has been determined among the automotive parts in the automotive body model, and generating an optimization analysis model having a sheet thickness of the automotive part in the automotive body model as a design variable: a sheet thickness optimization analysis condition setting step of setting an objective regarding a body mass of the optimization analysis model and a constraint regarding automotive body performance of the optimization analysis model as an optimization analysis condition for performing an optimization analysis of the sheet thickness of the automotive part in the optimization analysis model, and setting a load and constraint condition given to the optimization analysis model; and a sheet thickness optimization analysis step of performing the optimization analysis of the sheet thickness under the load and constraint condition and the optimization analysis condition set in the sheet thickness optimization analysis condition setting step, and obtaining an optimized sheet thickness of each of the automotive parts in the optimization analysis model.

The sensitivity analysis step may include calculating an element density of each element satisfying the objective under the constraint, and setting the calculated element density of each element as sensitivity of each element.

The automotive body model acquisition step may include setting all additional joining points capable of joining the parts assembly in addition to the joining points with respect to the acquired automotive body model.

An automotive body weight reduction apparatus reduces a weight of an automotive body model including a plurality of automotive parts, and includes: an automotive body model acquisition unit configured to acquire the automotive body model including the plurality of automotive parts modeled by a plurality of elements and joining points for joining the plurality of automotive parts as a parts assembly: a sensitivity analysis unit configured to set an objective regarding automotive body performance of the automotive body model, a constraint regarding a volume of the automotive body model, and a load and constraint condition or only a loading condition given to the automotive body model, and obtain sensitivity of each element satisfying the objective under the load and constraint condition or only the loading condition and the constraint: an automotive part dividing position/integration determination unit configured to determine a dividing position of the automotive parts and/or the automotive parts to be integrated based on the sensitivity of each element: a sheet thickness optimization analysis model generation unit configured to divide and/or integrate the automotive parts for which the dividing position and/or integration has been determined among the automotive parts in the automotive body model, and generate an optimization analysis model having a sheet thickness of the automotive part in the automotive body model as a design variable: a sheet thickness optimization analysis condition setting unit configured to set an objective regarding a body mass of the optimization analysis model and a constraint regarding automotive body performance of the optimization analysis model as an optimization analysis condition for performing an optimization analysis of the sheet thickness of the automotive part in the optimization analysis model, and set a load and constraint condition given to the optimization analysis model; and a sheet thickness optimization analysis unit configured to perform the optimization analysis of the sheet thickness under the load and constraint condition and the optimization analysis condition set by the sheet thickness optimization analysis condition setting unit, and obtain an optimized sheet thickness of the automotive parts in the optimization analysis model.

The sensitivity analysis unit may be configured to calculate an element density of each element satisfying the objective under the constraint, and set the calculated element density of each element as sensitivity of each element.

The automotive body model acquisition unit may be configured to set all additional joining points capable of joining the parts assembly in addition to the joining points with respect to the acquired automotive body model.

The sensitivity with respect to the automotive body performance is obtained for each element used for modeling the automotive part, the automotive parts to be divided and integrated are determined based on the obtained sensitivity of each element in the automotive parts, and optimization analysis of a sheet thickness is performed on an optimization analysis model having the automotive parts divided or integrated again according to the determination so that the automotive parts can be divided and integrated to reduce the weight of the automotive body, an optimized sheet thickness of each automotive part can be obtained, and the weight of the automotive body can be efficiently and sufficiently reduced while maintaining the automotive body performance.

### BRIEF DESCRIPTION OF THE DRAWINGS

FIG. 1 is a block diagram of an automotive body weight reduction apparatus according to an example.

FIG. 2 is a diagram illustrating an automotive body model, which is an analysis target, in the example.

FIGS. 3(a) and 3(b) are diagrams illustrating joining points and all additional joining points capable of joining in the automotive body model, which is an analysis target, in the example ((a) joining points and (b) joining points and all additional joining points capable of joining).

FIG. 4 is a diagram illustrating an example of a load and constraint condition applied to the automotive body model.

FIGS. 5(a)-5(c) are diagrams illustrating an example in which a dividing position and integration of automotive parts are determined based on a result of sensitivity analysis of the automotive parts on a front side of the automotive body model and an element density calculated as sensitivity by the sensitivity analysis ((a) a side diagram of the front side of an original automotive body model given in advance, (b) element density obtained by sensitivity analysis, and (c) a side diagram of the front side of an optimization analysis model generated by dividing and integrating again automotive parts).

FIGS. 6(a)-6(c) are diagrams illustrating an example in which dividing positions and integration of automotive parts are determined based on a result of sensitivity analysis of the automotive parts on a rear side of the automotive body model and an element density calculated as sensitivity by the sensitivity analysis ((a) a top diagram of the rear side of an original automotive body model given in advance, (b) element density obtained by sensitivity analysis, and (c) a top diagram of the rear side of an optimization analysis model generated by dividing and integrating again automotive parts).

FIGS. 7(a)-7(c) are diagrams illustrating an example in which dividing positions and integration of automotive parts are determined based on a result of sensitivity analysis of the automotive parts on a left side of the automotive body model and an element density obtained as sensitivity by the sensitivity analysis ((a) a perspective diagram of the left side of an original automotive body model given in advance, (b) element density obtained by sensitivity analysis, and (c) a perspective diagram of the left side of an optimization analysis model generated by dividing and integrating again automotive parts).

FIGS. 8(a) and 8(b) are diagrams illustrating an example of an optimization analysis model regenerated by dividing and integrating automotive parts ((a) original automotive body model given in advance, and (b) regenerated optimization analysis model).

FIG. 9 is a flowchart illustrating a flow of processing of an automotive body weight reduction method according to the example.

FIGS. 10(a)-10(c) are diagrams illustrating an example in which dividing positions and integration of automotive parts are determined based on a result of sensitivity analysis of the automotive parts on a front side of the automotive body model and an element density obtained as sensitivity by the sensitivity analysis in another aspect ((a) a side diagram of the front side of an original automotive body model given in advance, (b) element density obtained by sensitivity analysis, and (c) a side diagram of the front side of an optimization analysis model generated by dividing and integrating again).

FIGS. 11(a)-11(c) are diagrams illustrating an example in which dividing positions and integration of automotive parts are determined based on a result of sensitivity analysis of the automotive parts on a rear side of the automotive body model and an element density obtained as sensitivity by the sensitivity analysis in another aspect ((a) a top diagram of the rear side of an original automotive body model given in advance, (b) element density obtained by sensitivity analysis, and (c) a top diagram of the rear side of an optimization analysis model regenerated by dividing and integrating).

FIGS. 12(a)-12(c) are diagrams illustrating an example in which dividing positions and integration of automotive parts are determined based on a result of sensitivity analysis of the automotive parts on a left side of the automotive body model and an element density obtained as sensitivity by the sensitivity analysis in another aspect ((a) a perspective diagram of the left side of an original automotive body model given in advance, (b) element density obtained by sensitivity analysis, and (c) a perspective diagram of the left side of an optimization analysis model generated by dividing and integrating again).

FIGS. 13(a) and 13(b) are diagrams illustrating an example of an optimization analysis model generated by dividing and integrating automotive parts in another aspect ((a) original automotive body model given in advance, and (b) regenerated optimization analysis model).

### REFERENCE SIGNS LIST

1 WEIGHT REDUCTION APPARATUS
3 DISPLAY DEVICE
5 INPUT DEVICE
7 MEMORY STORAGE
9 WORKING DATA MEMORY
11 ARITHMETIC PROCESSING UNIT
13 AUTOMOTIVE BODY MODEL ACQUISITION UNIT
15 SENSITIVITY ANALYSIS UNIT
17 AUTOMOTIVE PART DIVIDING POSITION/INTEGRATION DETERMINATION UNIT
19 SHEET THICKNESS OPTIMIZATION ANALYSIS MODEL GENERATION UNIT
21 SHEET THICKNESS OPTIMIZATION ANALYSIS CONDITION SETTING UNIT
23 SHEET THICKNESS OPTIMIZATION ANALYSIS UNIT
25 AUTOMOTIVE BODY MODEL FILE
100 AUTOMOTIVE BODY MODEL
101 A-PILLAR LOWER
103 A-PILLAR UPPER
105 REAR ROOF RAIL CENTER
107 REAR ROOF RAIL SIDE
109 COMPARTMENT CENTER A
111 COMPARTMENT SIDE A
113 COMPARTMENT CENTER B
115 COMPARTMENT SIDE B
117 SIDE SILL OUTER
119 WHEEL HOUSE REINFORCEMENT
121 JOINING POINT
150 AUTOMOTIVE BODY MODEL
151 ALL ADDITIONAL JOINING POINTS CAPABLE OF JOINING
200 OPTIMIZATION ANALYSIS MODEL
201 A-PILLAR
203 REAR ROOF RAIL
205 COMPARTMENT A
207 COMPARTMENT B
209 SIDE SILL OUTER FRONT
211 SIDE SILL OUTER REAR
300 OPTIMIZATION ANALYSIS MODEL
301 A-PILLAR LOWER
303 A-PILLAR UPPER
305 REAR ROOF RAIL
307 COMPARTMENT A
309 COMPARTMENT B
311 WHEEL HOUSE REINFORCEMENT

### DETAILED DESCRIPTION

Prior to describing an example, an automotive body model will be described.

### Automotive Body Model

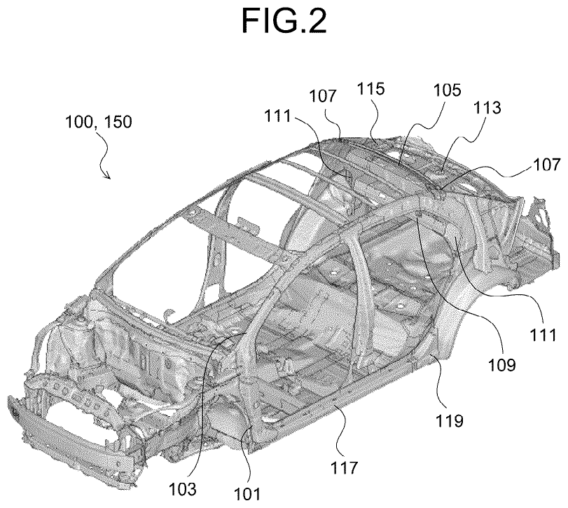

An automotive body model 100 to which my methods and apparatus are directed includes a plurality of automotive parts as illustrated in FIG. 2 as an example. Examples of the automotive parts include body frame parts such as an A-pillar lower 101, an A-pillar upper 103, a rear roof rail center 105, a rear roof rail side 107, a compartment center A 109, a compartment side A 111, a compartment center B 113, a compartment side B 115, a side sill outer 117, and a wheel house reinforcement 119, undercarriage parts (not illustrated) such as suspension parts and the like. Then, these automotive parts are modeled by a plurality of shell elements and/or solid elements.

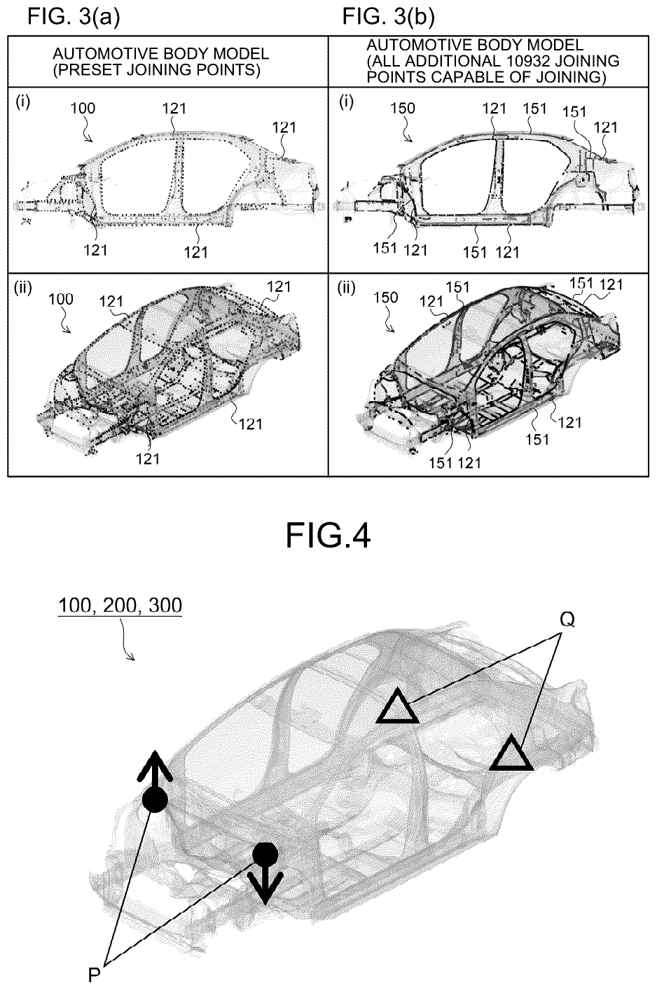

Further, in the automotive body model 100, as illustrated in FIG. 3(a) as an example, joining points 121 for joining a plurality of automotive parts as a parts assembly are set at predetermined intervals. Note that in the automotive body model 100, the interval between the joining points 121 is 25 to 60 mm.

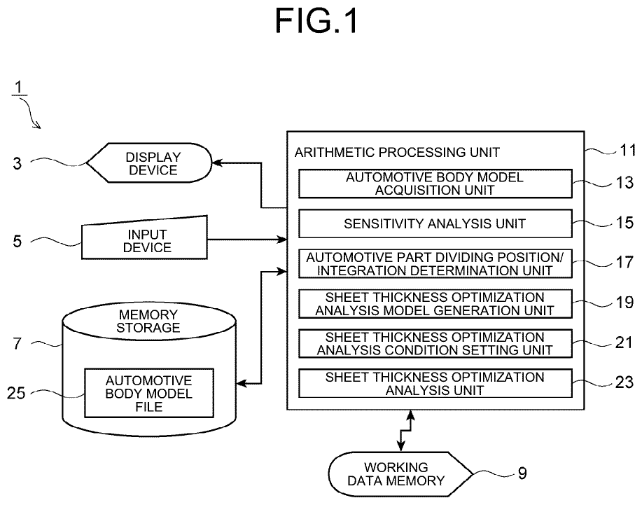

The material property and element information of each automotive part constituting the automotive body model 100, and furthermore information regarding the joining points 121 (FIG. 3(a)) and the like in each parts assembly are stored in an automotive body model file 25 (see FIG. 1) to be described below.

### Weight Reduction Apparatus

A configuration of a weight reduction apparatus that reduces the weight of an automotive body model according to an example will be described below.

A weight reduction apparatus 1 reduces the weight of an automotive body model, the automotive body model including a plurality of automotive parts. As illustrated in FIG. 1, the weight reduction apparatus 1 is configured by a personal computer (PC) or the like, and includes a display device 3, an input device 5, a memory storage 7, a working data memory 9, and an arithmetic processing unit 11. Then, the display device 3, the input device 5, the memory storage 7, and the working data memory 9 are connected to the arithmetic processing unit 11, and the respective functions are executed by a command from the arithmetic processing unit 11.

Hereinafter, each configuration of the weight reduction apparatus 1 according to the example will be described with respect to when the automotive body model 100 illustrated in FIGS. 2 and 3(a) and(b) is set as an analysis target, and the automotive parts are divided and integrated based on a result of the sensitivity analysis to obtain an optimized sheet thickness.

### Display Device

The display device 3 is used to display an analysis result or the like, and includes an LCD monitor or the like.

### Input Device

The input device 5 is used to instruct displays of the automotive body model file 25, input of conditions by an operator and the like, and includes a keyboard, a mouse and the like.

### Memory Storage

The memory storage 7 is used to store various files such as the automotive body model file 25 in which various types of information regarding an automotive body model are recorded as described below, and includes a hard disk or the like.

### Working Data Memory

The working data memory 9 is used for temporary storage and calculation of data used by the arithmetic processing unit 11, and includes random access memory (RAM) or the like.

### Arithmetic Processing Unit

As illustrated in FIG. 1, the arithmetic processing unit 11 includes an automotive body model acquisition unit 13, a sensitivity analysis unit 15, an automotive part dividing position/integration determination unit 17, a sheet thickness optimization analysis model generation unit 19, a sheet thickness optimization analysis condition setting unit 21, and a sheet thickness optimization analysis unit 23, and is configured by a central processing unit (CPU) such as of a PC. Each of these units functions when the CPU executes a predetermined program. The functions of the units described above in the arithmetic processing unit 11 will be described below.

### Automotive Body Model Acquisition Unit

The automotive body model acquisition unit 13 acquires the automotive body model 100 including automotive parts (A-pillar lower 101 and the like) modeled by a plurality of elements and joining points 121 to join the plurality of automotive parts as a parts assembly as illustrated in FIGS. 2 and 3(a).

In this example, each automotive part constituting the automotive body model 100 is assumed to be modeled by a shell element as an example, and information regarding the shell element constituting each automotive part and material property (Young's modulus, specific gravity, Poisson's ratio, or the like) of each automotive part is recorded in the automotive body model file 25 (see FIG. 1) stored in the memory storage 7. Therefore, the automotive body model acquisition unit 13 can acquire the automotive body model 100 by reading the automotive body model file 25.

### Sensitivity Analysis Unit

The sensitivity analysis unit 15 sets objectives regarding the automotive body performance of the automotive body model 100, constraints regarding the volume of the automotive body model 100, and a load and constraint condition or only a loading condition given to the automotive body model 100, and obtains the sensitivity of each element in each automotive part satisfying the objectives under the set load and constraint condition or only loading condition and the constraints.

In this example, the objectives regarding the automotive body performance set by the sensitivity analysis unit 15 include minimization of the sum of strain energy, minimization of displacement, minimization of stress, maximization of stiffness and the like in the automotive body model 100, and it is sufficient if these objectives are appropriately selected according to the target automotive body performance.

In addition, as the constraints regarding the volume of the automotive body model 100 set by the sensitivity analysis unit 15, there is a volume fraction ratio that defines the volume of the automotive part and the like.

As the load and constraint condition set in the automotive body model 100 by the sensitivity analysis unit 15, for example, the load and constraint condition illustrated in FIG. 4 is set. The load and constraint condition illustrated in FIG. 4 is obtained by setting left and right front suspension attachment positions (P in the drawing) of the automotive body model 100 as load points, applying a vertically upward load to one of the attachment positions and a vertically downward load to the other of the attachment positions, and further constraining left and right rear subframe attachment positions (Q in the drawing) of the automotive body model 100.

Further, the sensitivity analysis unit 15 preferably calculates the element density of each element as the sensitivity of each element in each automotive part by using topology optimization in which densimetry is applied. The element density of each element calculated at this time corresponds to density p indicated in Formula (1):

$$F = ρ Kx \qquad ( 1 )$$

F: Load vector
ρ: Normalized density
K: Stiffness matrix
x: Displacement vector

The normalized density p in Formula (1) is a virtual density indicating the filling state of the material in each element, and takes a value from 0 to 1. That is, when the element density p of the element is 1, it indicates a state in which the element is fully filled with the material, when the element density p is 0, it indicates a state in which the element is not filled with the material and is fully hollow, and when the element density of the element is an intermediate value between 0 and 1, it indicates an intermediate state in which the element is neither a material nor hollow.

Then, the element density calculated by the topology optimization indicates that the element density of the element having a large contribution to the automotive body performance becomes a value close to 1, and the sensitivity with respect to the automotive body performance is high. On the other hand, regarding an element having a small contribution to the automotive body performance, the element density of the element becomes a value close to 0, which indicates that the sensitivity with respect to the automotive body performance is low. As described above, the element density of the element calculated by topology optimization is an index indicating the sensitivity of each element with respect to the automotive body performance.

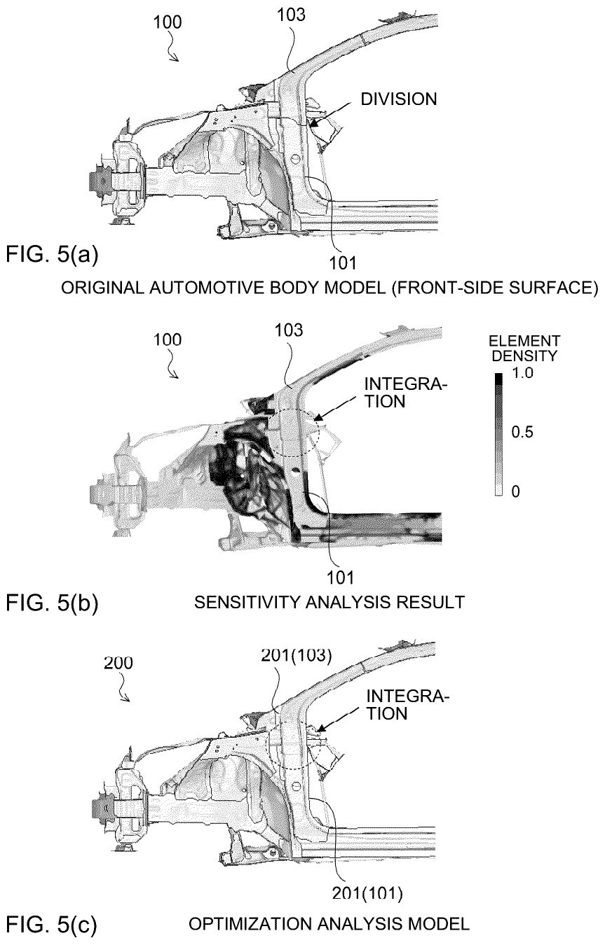

As an example of the sensitivity of the element calculated by the sensitivity analysis unit 15, FIGS. 5(b), 6(b), and 7(b) illustrate an example of the result of the element density calculated for the element of each automotive part when the objective is maximization of stiffness, the constraint is a volume fraction ratio of 25%, and static torsion is applied to the automotive body model 100 according to the load and constraint condition (absolute value 1000 N of the load applied to the load point) illustrated in FIG. 4.

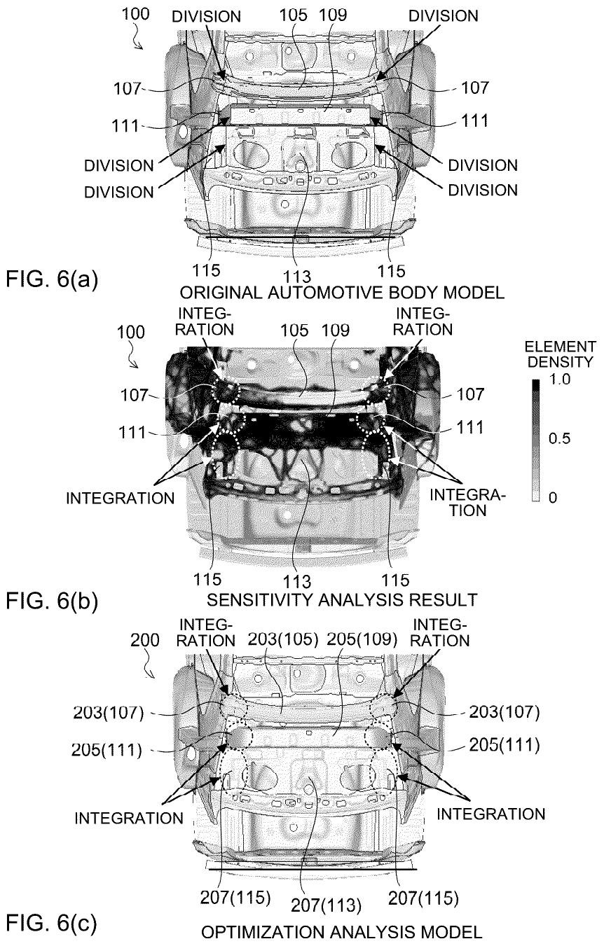

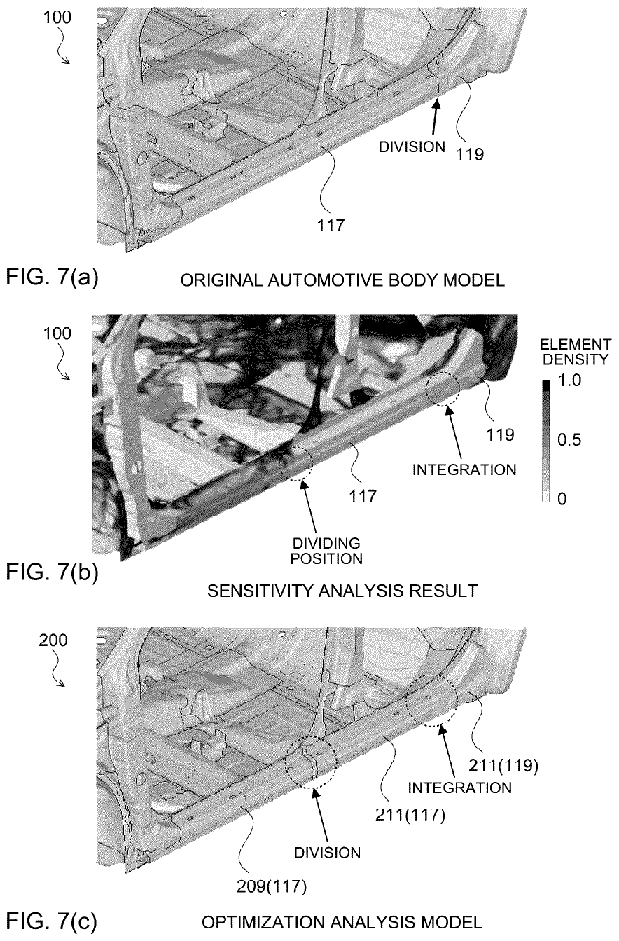

FIG. 5(b) is a side diagram of the A-pillar lower 101 and the A-pillar upper 103 (FIG. 5(a)) on the front side of the automotive body model 100, FIG. 6(b) is a top diagram on the rear side (FIG. 6(a)) of the automotive body model 100, and FIG. 7(b) is a perspective diagram of the side sill outer 117 and the wheel house reinforcement 119 (FIG. 7(a)) on the left side of the automotive body model 100.

As illustrated in FIGS. 5(b), 6(b), and 7(b), it can be seen that there are a region having high sensitivity to static torsion and a region having low sensitivity even in the same automotive part (for example, the side sill outer 117 illustrated in FIG. 7(b)), and that is when the sensitivity is substantially the same as a whole even in different automotive parts (for example, the A-pillar lower 101 and the A-pillar upper 103 illustrated in FIG. 5(b)).

The sensitivity analysis unit 15 may set only a loading condition considering an inertia force when a dynamic load is applied to the automotive body model 100 by an inertia relief method. The inertia relief method is an analysis method for obtaining stress and strain from force acting on an object during constant acceleration motion in a state where the object is supported at a support point serving as a reference of coordinates of inertial force (free support), and is used for static analysis of an airplane or a ship in motion.

In addition, in calculating the element density of the element with the sensitivity analysis unit 15, analysis software for performing optimization analysis such as topology optimization can be used. In this example, each automotive part constituting the automotive body model 100 is set as a design space, the element density is given as a design variable to the element constituting the automotive part set as the design space, and predetermined objective and constraint and load and constraint condition are set so that the element density is calculated as the sensitivity of the element.

However, when the optimization analysis is performed in the sensitivity analysis unit 15, an optimization analysis method other than the topology optimization may be applied.

### Automotive Part Dividing Position/Integration Determination Unit

The automotive part dividing position/integration determination unit 17 determines a dividing position and/or integration of the automotive parts according to an instruction of the operator based on the sensitivity of each element in the automotive part obtained by the sensitivity analysis unit 15.

In determining the dividing position of the automotive parts and the automotive parts to be integrated based on the sensitivity, it is sufficient if the difference in sensitivity is used as an index, a position where the difference in sensitivity is large in the same automotive part may be determined as the dividing position and adjacent automotive parts having a small difference in sensitivity are determined to be integrated according to an instruction of the operator.

In this example, the position where the difference in sensitivity in the automotive parts is 0.7 or more is determined as the dividing position, and integration is determined when the difference in sensitivity between adjacent automotive parts is 0.3 or less.

FIGS. 5(b), 6(b), and 7(b) illustrate element densities of the automotive parts on the front side, the rear side, and the left side of the automotive body model 100 obtained by the sensitivity analysis, and an example of a result of determining the dividing positions of the automotive parts and the automotive parts to be integrated based on the element densities.

On the front side (FIG. 5(a)) of the automotive body model 100, as illustrated in FIG. 5(b), the difference in sensitivity (element density) between the A-pillar lower 101 and the A-pillar upper 103 was as small as 0.3 or less (broken-line ellipse in the drawing). Hence, the automotive part dividing position/integration determination unit 17 determines to integrate the A-pillar lower 101 and the A-pillar upper 103 according to an instruction of the operator.

On the rear side (FIG. 6(a)) of the automotive body model 100, as indicated by the broken line ellipses in FIG. 6(b), the difference in sensitivity between the rear roof rail center 105 and the rear roof rail side 107, between the compartment center A 109 and the compartment side A 111, and between the compartment center B 113 and the compartment side B 115 was as small as 0.3 or less. Hence, the automotive part dividing position/integration determination unit 17 determines to integrate the rear roof rail center 105 and the rear roof rail side 107, the compartment center A 109 and the compartment side A 111, and the compartment center B 113 and the compartment side B 115 according to an instruction of the operator.

On the left side (FIG. 7(a)) of the automotive body model 100, as indicated by the broken line ellipses in FIG. 7(b), the difference in sensitivity between the front side and the rear side from a substantially center of the side sill outer 117 was as large as 0.7 or more, and the difference in sensitivity between a rear portion of the side sill outer 117 and the wheel house reinforcement 119 was as small as 0.3 or less. Hence, the automotive part dividing position/integration determination unit 17 determines, as the dividing position, the substantially center having a large difference in sensitivity in the side sill outer 117 according to an instruction of the operator.

In this example, the position where the difference in sensitivity is 0.7 or more in the automotive parts is determined as the dividing position, and the adjacent automotive parts where the difference in sensitivity is 0.3 or less are determined to be integrated, but the difference in sensitivity for determining the dividing position or the integration may be appropriately selected.

### Sheet Thickness Optimization Analysis Model Generation Unit

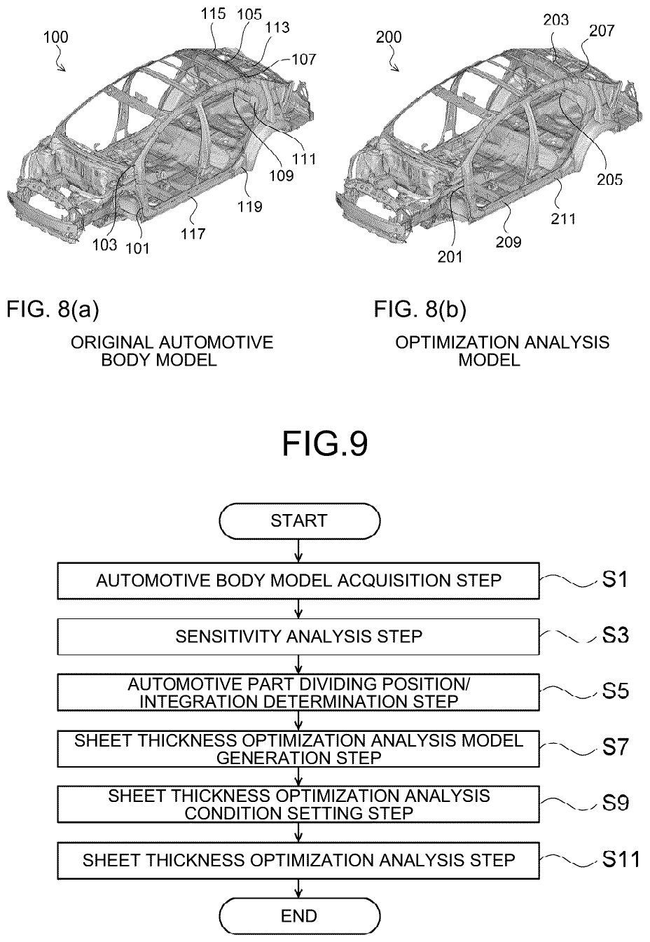

The sheet thickness optimization analysis model generation unit 19 divides and/or integrates again the automotive parts the dividing positions and/or integration of which has been determined by the automotive part dividing position/integration determination unit 17 among the automotive parts in the automotive body model 100 as illustrated in FIG. 8(a), and generates an optimization analysis model 200 having the sheet thickness of the automotive part as a design variable as illustrated in FIG. 8(b).

FIGS. 5(c), 6(c), and 7(c) illustrate the automotive parts on the front side, the rear side, and the left side of the optimization analysis model 200. In addition, FIG. 8(b) illustrates an overall diagram of the optimization analysis model 200.

On the front side of the automotive body model 100, according to the determination to integrate the automotive parts illustrated in FIG. 5(b), as illustrated in FIG. 5(c), the A-pillar lower 101 and the A-pillar upper 103 are integrated to form an A-pillar 201.

On the rear side of the automotive body model 100, according to the determination to integrate the automotive parts illustrated in FIG. 6(b), as illustrated in FIG. 6(c), the rear roof rail center 105 and the rear roof rail side 107 are integrated to form a rear roof rail 203, the compartment center A 109 and the compartment side A 111 are integrated to form a compartment A 205, and the compartment center B 113 and the compartment side B 115 are integrated to form a compartment B 207.

On the left side of the automotive body model 100, according to the determination of the dividing position and integration of the automotive parts illustrated in FIG. 7(b), as illustrated in FIG. 7(c), the front side of the side sill outer 117 is divided to be a side sill outer front 209, and the rear side of the side sill outer 117 from the dividing position is integrated with the wheel house reinforcement 119 to form a side sill outer rear 211. In the optimization analysis model 200, the sheet thickness of the automotive parts after the division remains the sheet thickness of the automotive parts before the division, and the integrated automotive parts have the sheet thickness of the automotive part having the larger surface area among the automotive parts before the integration.

### Sheet Thickness Optimization Analysis Condition Setting Unit

The sheet thickness optimization analysis condition setting unit 21 sets the objective regarding the body mass of the optimization analysis model 200 and the constraint regarding the automotive body performance of the optimization analysis model 200 and sets the load and constraint condition to be given to the optimization analysis model 200 as the optimization analysis condition for performing the optimization analysis of the sheet thickness of the automotive parts in the optimization analysis model 200.

Only one objective is set according to the purpose of the optimization analysis. In this example, minimization of the body mass is set as an objective.

The constraints are constraints imposed when the optimization analysis is performed, and a plurality of constraints is set as necessary. In this example, a constraint regarding the automotive body performance and a constraint regarding the sheet thickness of the automotive parts are set.

As the constraint regarding the automotive body performance, the stiffness of the optimization analysis model is equal to or more than a predetermined stiffness, and as the predetermined stiffness, for example, it is sufficient if the stiffness of the original automotive body model 100 before the optimization analysis of the sheet thickness is performed is provided. Regarding the stiffness of the optimization analysis model 200 and the automotive body model 100, for example, displacement or strain of a load point is preferably used as an index.

In addition, as the constraint regarding the sheet thickness of the automotive parts, a constraint to select from a plurality of sheet thicknesses of a steel sheet generally used for manufacturing the automotive parts is set instead of a continuously changing value of the sheet thickness. In this example, a constraint to select from 0.55 mm, 0.60 mm, 0.65 mm, 0.70 mm, 0.75 mm, 0.80 mm, 0.85 mm, 0.90 mm, 1.0 mm, 1.2 mm, 1.4 mm, 1.6 mm, 1.8 mm, 2.0 mm, 2.3 mm, 2.6 mm, 3.2 mm, 3.4 mm, 3.6 mm, and 4.0 mm, which are sheet thicknesses of a steel sheet generally used to manufacture the automotive parts, is set.

The load and constraint condition is a condition regarding a load (position, size, direction) and a constraint position given to the optimization analysis model in the optimization analysis of the sheet thickness. In this example, the load and constraint condition is to set left and right front suspension attachment positions (P in FIG. 4) of the optimization analysis model 200 as load points, apply a vertically upward load to one of the attachment positions and a vertically downward load to the other of the attachment positions, and further constrain left and right rear subframe attachment positions (Q in FIG. 4) of the optimization analysis model 200.

### Sheet Thickness Optimization Analysis Unit

The sheet thickness optimization analysis unit 23 performs optimization analysis of the sheet thickness under the load and constraint condition and the optimization analysis condition set by the sheet thickness optimization analysis condition setting unit 21, and obtains the optimized sheet thickness of each automotive part in the optimization analysis model 200.

As described above, in the optimization analysis of the sheet thickness, the sheet thickness of the optimization analysis model 200 is used as a design variable, and further the constraint regarding the sheet thickness is imposed. Therefore, the optimized sheet thickness is obtained for each automotive part from among the plurality of sheet thicknesses imposed as the constraint by the sheet thickness optimization analysis unit 23.

### Automotive Body Weight Reduction Method

An automotive body weight reduction method according to this example reduces the weight of an automotive body model, the automotive body model including a plurality of automotive parts, by performing the steps described below with a computer. As illustrated in FIG. 9, this method includes an automotive body model acquisition step S1, a sensitivity analysis step S3, an automotive part dividing position/integration determination step S5, a sheet thickness optimization analysis model generation step S7, a sheet thickness optimization analysis condition setting step S9, and a sheet thickness optimization analysis step S11. In this example, each of the steps described above is executed by the weight reduction apparatus 1 (see FIG. 1) configured by a computer. Each of the steps described above will be described below.

### Automotive Body Model Acquisition Step

The automotive body model acquisition step S1 is a step of acquiring an automotive body model including a plurality of automotive parts modeled by a plurality of elements and joining points for joining the plurality of automotive parts as a parts assembly. In this example, the automotive body model acquisition unit 13 of the weight reduction apparatus 1 reads the automotive body model file 25 (see FIG. 1) to acquire the automotive body model 100 including the plurality of automotive parts (A-pillar lower 101 and the like) modeled by a plurality of shell elements and the joining points 121 for joining the automotive parts as a parts assembly as illustrated in FIGS. 2 and 3(a) as an example.

### Sensitivity Analysis Step

The sensitivity analysis step S3 is a step of setting an objective regarding the automotive body performance of the automotive body model 100, a constraint regarding the volume of the automotive body model 100, and a load and constraint condition or only a loading condition given to the automotive body model 100, and obtaining the sensitivity of each element in each automotive part satisfying the objective under the set load and constraint condition or only loading condition and the constraint. In this example, the sensitivity analysis unit 15 of the weight reduction apparatus 1 sets the objective, the constraint, and the load and constraint condition, and calculates the element density of each element as the sensitivity of each element.

In the sensitivity analysis step S3, optimization analysis such as topology optimization may be performed. In this example, it is sufficient if the automotive parts constituting the automotive body model 100 are set as the design space, the element density is given as the design variable to the element constituting the automotive parts set as the design space, the optimization analysis processing is executed, and the element density satisfying the objective under the set constraint and load and constraint condition is calculated for each element in the automotive part.

### Automotive Part Dividing Position/Integration Determination Step

The automotive part dividing position/integration determination step S5 is a step of determining a dividing position and/or integration of the automotive parts with a computer according to an instruction of the operator based on the sensitivity of each element in the automotive part obtained in the sensitivity analysis step S3. In this example, this is performed by the automotive part dividing position/integration determination unit 17 of the weight reduction apparatus 1.

### Sheet Thickness Optimization Analysis Model Generation Step

The sheet thickness optimization analysis model generation step S7 is a step of dividing and/or integrating again the automotive parts the dividing positions and/or integration of which has been determined among the automotive parts in the automotive body model 100 as illustrated in FIGS. 5 to 8, and generating the optimization analysis model 200 having the sheet thickness of the automotive part of the automotive body model 100 as the design variable. In this example, this is performed by the sheet thickness optimization analysis model generation unit 19 of the weight reduction apparatus 1.

### Sheet Thickness Optimization Analysis Condition Setting Step

The sheet thickness optimization analysis condition setting step S9 is a step of setting the objective regarding the body mass of the optimization analysis model 200 and the constraint regarding the automotive body performance of the optimization analysis model 200 and setting the load and constraint condition to be given to the optimization analysis model 200 as the optimization analysis condition for performing the optimization analysis of the sheet thickness of the automotive parts in the optimization analysis model 200. In this example, this is performed by the sheet thickness optimization analysis condition setting unit 21 of the weight reduction apparatus 1.

### Sheet Thickness Optimization Analysis Step

The sheet thickness optimization analysis step S11 is a step of performing optimization analysis of the sheet thickness under the optimization analysis condition set in the sheet thickness optimization analysis condition setting step S9, and obtaining the optimized sheet thickness of each automotive part in the optimization analysis model 200. In this example, this is performed by the sheet thickness optimization analysis unit 23 of the weight reduction apparatus 1.

According to the method and apparatus for weight reduction of an automotive body according to this example, the sensitivity with respect to the automotive body performance is obtained for each element used for modeling the automotive part, the automotive parts to be divided and integrated are determined based on the obtained sensitivity of each element in the automotive parts, and the optimization analysis of the sheet thickness in which the automotive body performance is set as the constraint is performed on the optimization analysis model having the automotive parts divided or integrated again according to the determination. Thus, it is possible to obtain the optimized sheet thickness of each automotive part by dividing and integrating again the automotive parts to reduce the weight of the automotive body, and it is possible to efficiently and sufficiently reduce the weight of the automotive body while maintaining the automotive body performance.

In the description described above, the sensitivity analysis is performed using the automotive body model 100 in which the joining points 121 are set as it is, and the dividing position of the automotive parts and the automotive parts to be integrated are determined, but there is an instance where the sensitivity with respect to the automotive body performance varies depending on a difference in the number of joining points 121 set in the automotive body model 100.

Hence, as another aspect of this example, as illustrated in FIG. 3(b) as an example, in addition to the joining points 121 having an interval of 25 to 60 mm between the joining points, all additional joining points 151 capable of joining the parts assembly may be set with respect to the acquired automotive body model 100 to dense joining points, and the sensitivity analysis may be performed using an automotive body model 150 simulating that the plurality of automotive parts is continuously joined. In the automotive body model 150, 10932 additional joining points 151 capable of joining are set at an interval of 10 mm.

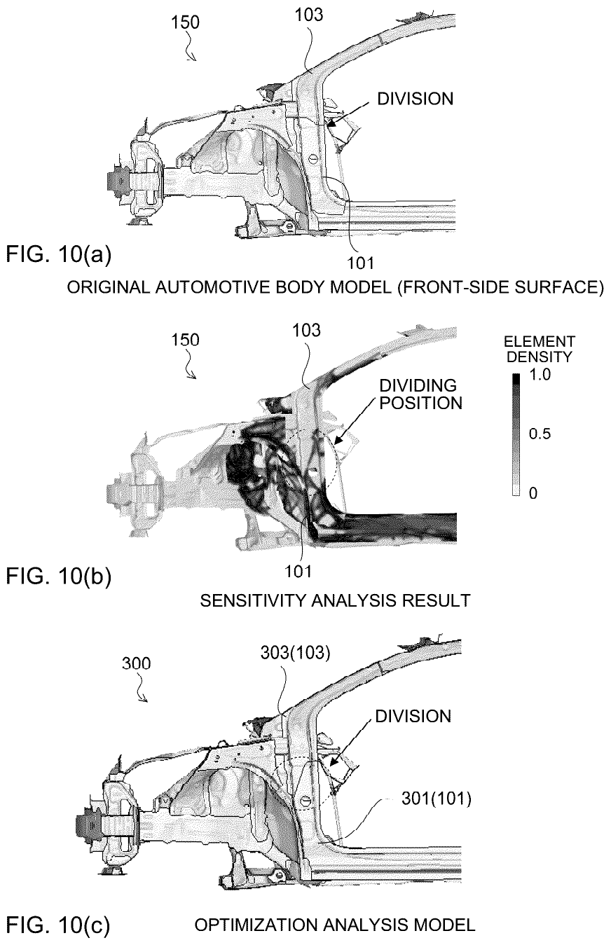

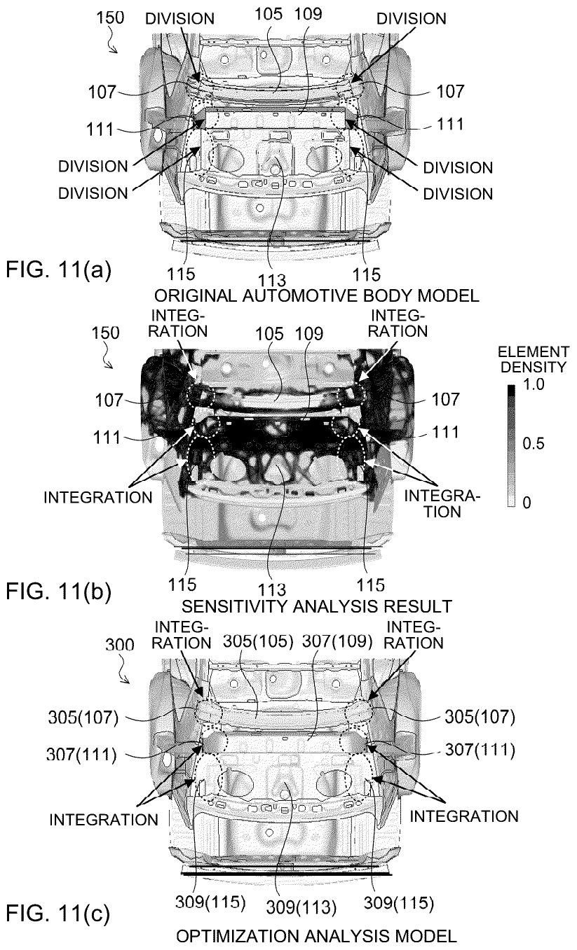

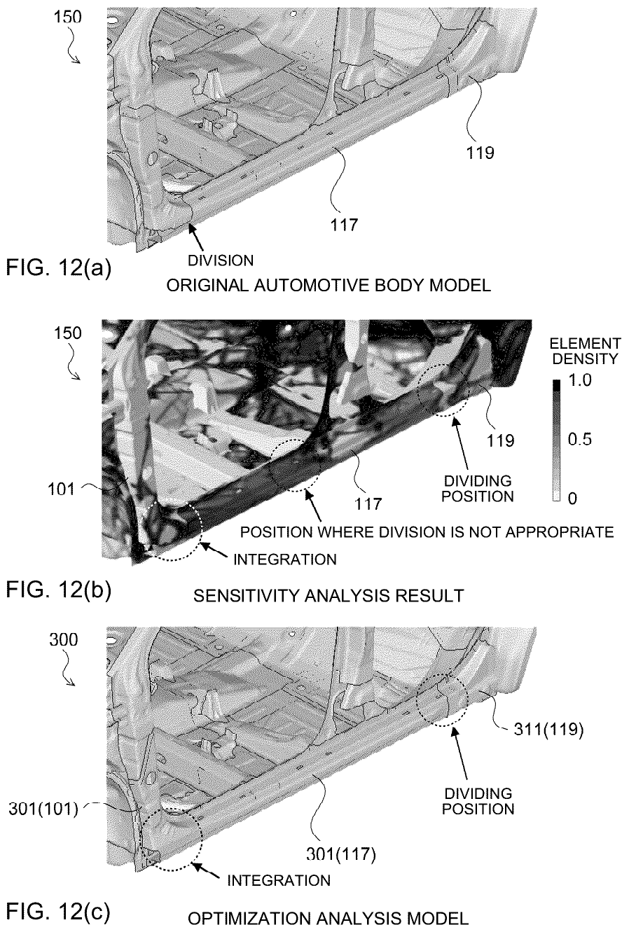

FIGS. 10(b), 11(b), and 12(b) illustrate results of the sensitivity analysis performed using the automotive body model 150 in which the 10932 additional joining points 151 capable of joining are set in the automotive body model 100, and the dividing positions of the automotive parts and the automotive parts to be integrated are determined. The automotive parts in the automotive body model 150 are denoted by the same reference numerals as the automotive parts in the automotive body model 100 illustrated in FIG. 2. Then, FIG. 10(b) is a side diagram of the A-pillar lower 101 and the A-pillar upper 103 (FIG. 10(a)) on the front side of the automotive body model 150, FIG. 11(b) is a top diagram on the rear side (FIG. 11(a)) of the automotive body model 150, and FIG. 12(b) is a perspective diagram of the side sill outer 117 and the wheel house reinforcement 119 (FIG. 12(a)) on the left side of the automotive body model 150. In addition, the sensitivities illustrated in FIGS. 10(b), 11(b), and 12(b) are obtained by setting the same objective, constraint, and load and constraint condition (see FIG. 4) as those of this example described above.

On the front side (FIG. 10(a)) of the automotive body model 150, as illustrated in FIG. 10(b), the difference in sensitivity was as large as 0.7 or more at a position different from the boundary between the A-pillar lower 101 and the A-pillar upper 103. Therefore, the position where the difference in sensitivity is large is determined as the dividing position, and as illustrated in FIG. 10(c), is newly divided into an A-pillar lower 301 and an A-pillar upper 303.

On the rear side (FIG. 11(a)) of the automotive body model 150, as illustrated in FIG. 11(b), the difference in sensitivity between the rear roof rail center 105 and the rear roof rail side 107, between the compartment center A 109 and the compartment side A 111, and between the compartment center B 113 and the compartment side B 115 was as small as 0.3 or less.

Therefore, it is determined that the automotive parts having a small difference in sensitivity are integrated, and as illustrated in FIG. 11(c), the rear roof rail center 105 and the rear roof rail side 107 are integrated to form a rear roof rail 305, the compartment center A 109 and the compartment side A 111 are integrated to form a compartment A 307, and the compartment center B 113 and the compartment side B 115 are integrated to form a compartment B 309.

On the left side (FIG. 12(a)) of the automotive body model 150, as illustrated in FIG. 12(b), the difference in sensitivity in the side sill outer 117 was as small as 0.3 or less, and the difference in sensitivity between the rear portion of the side sill outer 117 and the wheel house reinforcement 119 was as large as 0.7 or more. Further, the difference in sensitivity between the A-pillar lower 101 and a front portion of the side sill outer 117 was as small as 0.3 or less.

Therefore, it is determined that the side sill outer 117 is integrated with the Apillar lower 101 without being divided, and further the side sill outer 117 and the wheel house reinforcement 119 are not integrated but are remained divided, and as illustrated in FIG. 12(c), the side sill outer 117 is integrated with the A-pillar lower 101 to form the A-pillar lower 301, and the wheel house reinforcement 119 is not integrated with the side sill outer 117 to form a wheel house reinforcement 311.

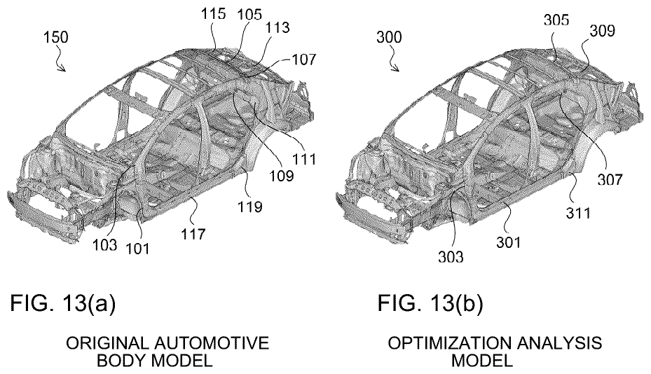

FIG. 13(b) illustrates an overall diagram of an optimization analysis model 300 generated by determining dividing positions and integration of the automotive parts based on the sensitivities illustrated in FIGS. 10(b), 11(b), and 12(b) and dividing and integrating again the automotive parts based on the determination.

The difference in operation and effect between the instance when the automotive body model 100 in which the joining points 121 are set is used as it is described as this example and when the automotive body model 150 in which all the additional joining points 151 capable of joining are further set is used described as another aspect of this disclosure will be described in an example described below.

In addition, the sensitivity analysis unit 15 and the sensitivity analysis step S3 in this example calculate the element density for each element as the sensitivity of each element. However, when an automotive part is modeled by a plurality of shell elements, the sheet thickness of each shell element satisfying predetermined objective, constraint, and load and constraint condition may be calculated, and the calculated sheet thickness of the shell element may be used as the sensitivity of each element.

As described above, when the sheet thickness of each shell element obtained in the sensitivity analysis is used as the sensitivity, the element having a large sheet thickness indicates that the sensitivity with respect to the automotive body performance is high, and the shell element having a small sheet thickness indicates that the sensitivity with respect to the automotive body performance is low. Thus, the sheet thickness of the element calculated in the sensitivity analysis can be an index indicating the sensitivity of each element with respect to the automotive body performance.

Further, in this example, the sensitivity analysis unit 15 and the sensitivity analysis step S3 perform the sensitivity analysis by setting a load and constraint condition for giving a static load, but this example may set a load and constraint condition corresponding to a dynamic load for vibrating the automotive body.

Specifically, frequency response analysis or the like is performed on the automotive body model prior to the sensitivity analysis, and the position, direction, and magnitude of the load to be applied to the automotive body model corresponding to the deformation state in the vibration mode of the automotive body model obtained by the frequency response analysis or the like are determined. Then, it is sufficient if the determined position, the direction, and the magnitude of the load to be applied are set as the load and constraint condition, and the sensitivity analysis is performed.

### EXAMPLE

An experiment verifying the desired effects of the method and apparatus for weight reduction of an automotive body has been performed, and this will be described below.

In this example, as an inventive example, as described in the above-described example, the optimization analysis of the sheet thickness was performed on the optimization analysis model 200 (FIG. 8(b)) and the optimization analysis model 300 (FIG. 12(b)) regenerated by dividing and integrating the automotive parts based on the sensitivity obtained for each element of each automotive part by the sensitivity analysis with respect to the automotive body performance, and the effects of the automotive body weight reduction with respect to the automotive body model 100 before dividing and integrating the automotive parts were verified.

In the optimization analysis of the sheet thickness, as the load and constraint condition, as illustrated in FIG. 4, the left and right front suspension attachment positions (P in the drawing) of the optimization analysis model 200 and the optimization analysis model 300 were set as load points, a vertically upward load (1000 N) was applied to one of the attachment positions and a vertically downward load (1000 N) was applied to the other of the attachment positions, and further the left and right rear subframe attachment positions (Q in the drawing) of the automotive body model 100 were constrained.

Further, as the optimization analysis condition, the objective to minimize the body mass, and the constraint to have the stiffness or more of the original automotive body model 100 given in advance and to select the sheet thickness of the steel sheet used for the automotive part from 0.55 mm, 0.60 mm, 0.65 mm, 0.70 mm, 0.75 mm, 0.80 mm, 0.85 mm, 0.90 mm, 1.0 mm, 1.2 mm, 1.4 mm, 1.6 mm, 1.8 mm, 2.0 mm, 2.3 mm, 2.6 mm, 3.2 mm, 3.4 mm, 3.6 mm, and 4.0 mm were set.

In addition, in this example, the optimization analysis of the sheet thickness was also performed on the original automotive body model 100 given in advance as a comparison target, similarly to the inventive example. Table 1 indicates the weight reduction effect by the optimization analysis of the sheet thickness of the automotive body model 100, the optimization analysis model 200, and the optimization analysis model 300.

TABLE 1

| | Body mass before | Body mass after | | Weight reduction |
|---|---|---|---|---|
| | optimization of | optimization of | Weight reduction | amount by division |
| | sheet thickness | sheet thickness | amount of body | and integration of |
| | (kg) | (kg) | mass (kg) | automotive parts (kg) |
| Comparative | 299.8 | 267.6 | 32.23 | — |
| example | | | | |
| First inventive | 302.1 | 262.7 | 37.08 | 4.85 |
| example | | | | |
| Second inventive | 301.4 | 262.0 | 37.79 | 5.56 |
| example | | | | |

In Table 1, in a comparative example, the optimization analysis of the sheet thickness was performed for the automotive body model 100, in a first inventive example, the optimization analysis of the sheet thickness was performed for the optimization analysis model 200, and in a second inventive example, the optimization analysis of the sheet thickness was performed for the optimization analysis model 300, and for each of the above, the body mass before the optimization of the sheet thickness, the body mass after the optimization analysis of the sheet thickness, and the weight reduction amount of the body mass by the optimization analysis of the sheet thickness are indicated. Further, regarding the first and second inventive examples, the weight reduction amount by the division and integration of the automotive parts is indicated. The weight reduction amount by the division and integration of the automotive parts was calculated by the equation described below:

(Weight reduction amount by division and integration of automotive parts)=(weight reduction amount of body mass in first inventive example or second inventive example)−(weight reduction amount of body mass in comparative example).

As indicated in Table 1, in any of the comparative example, the first inventive example, and the second inventive example, the body mass was greatly reduced by the optimization analysis of the sheet thickness, and in the first and second inventive examples, the weight reduction amounts of body mass result in 4.85 kg and 5.56 kg compared to the comparative example by the division and integration of the automotive parts. Given the above, it was indicated that the weight reduction effect of the automotive body can be further obtained while maintaining the automotive body performance by dividing and integrating again the automotive parts based on the sensitivity of the element with respect to the automotive body performance.

Further, in the second inventive example using the optimization analysis model 300 in which all the additional joining points 151 (FIG. 3(b)) capable of joining are set and the automotive parts are divided and integrated again, the weight reduction amount results in 13% larger than that in the first inventive example using the optimization analysis model 200 in which all the additional joining points 151 capable of joining are not set and the automotive parts are divided and integrated again. Accordingly, I found that it is preferable to perform the sensitivity analysis by densely setting all the additional joining points 151 capable of joining in the automotive body model 100, determine the dividing position of the automotive parts and the automotive parts to be integrated, and perform the optimization analysis of the sheet thickness by dividing and integrating the automotive parts according to the determination.

### INDUSTRIAL APPLICABILITY

It is possible to provide a method and apparatus for weight reduction of an automotive body capable of efficiently and sufficiently reducing the weight of the automotive body while maintaining the performance of the automotive body.

1-6. (canceled)

7. An automotive body weight reduction method for reducing a weight of an automotive body model including a plurality of automotive parts, the method being executed by a computer and comprising:
an automotive body model acquisition step of acquiring the automotive body model including the plurality of automotive parts modeled by a plurality of elements and joining points for joining the plurality of automotive parts as a parts assembly;
a sensitivity analysis step of
setting an objective regarding automotive body performance of the automotive body model, a constraint regarding a volume of the automotive body model, and a load and constraint condition or only a loading condition given to the automotive body model, and
obtaining sensitivity of each element satisfying the objective under the load and constraint condition or only the loading condition and the constraint;
an automotive part dividing position/integration determination step of determining a dividing position of the automotive parts and/or the automotive parts to be integrated based on the sensitivity of each element;
a sheet thickness optimization analysis model generation step of
dividing and/or integrating the automotive parts for which the dividing position and/or integration has been determined among the automotive parts in the automotive body model, and
generating an optimization analysis model having a sheet thickness of the automotive part in the automotive body model as a design variable;
a sheet thickness optimization analysis condition setting step of
setting an objective regarding a body mass of the optimization analysis model and a constraint regarding automotive body performance of the optimization analysis model as an optimization analysis condition for performing an optimization analysis of the sheet thickness of the automotive part in the optimization analysis model, and
setting a load and constraint condition given to the optimization analysis model; and
a sheet thickness optimization analysis step of
performing the optimization analysis of the sheet thickness under the load and constraint condition and the optimization analysis condition set in the sheet thickness optimization analysis condition setting step, and
obtaining an optimized sheet thickness of each of the automotive parts in the optimization analysis model.

8. The method according to claim 7, wherein the sensitivity analysis step includes
calculating an element density of each element satisfying the objective under the constraint, and
setting the calculated element density of each element as sensitivity of each element.

9. The method according to claim 7, wherein the automotive body model acquisition step includes setting all additional joining points capable of joining the parts assembly in addition to the joining points with respect to the acquired automotive body model.

10. An automotive body weight reduction apparatus for reducing a weight of an automotive body model including a plurality of automotive parts, the apparatus comprising:
an automotive body model acquisition unit configured to acquire the automotive body model including the plurality of automotive parts modeled by a plurality of elements and joining points for joining the plurality of automotive parts as a parts assembly;
a sensitivity analysis unit configured to
set an objective regarding automotive body performance of the automotive body model, a constraint regarding a volume of the automotive body model, and a load and constraint condition or only a loading condition given to the automotive body model, and
obtain sensitivity of each element satisfying the objective under the load and constraint condition or only the loading condition and the constraint;
an automotive part dividing position/integration determination unit configured to determine a dividing position of the automotive parts and/or the automotive parts to be integrated based on the sensitivity of each element;
a sheet thickness optimization analysis model generation unit configured to
divide and/or integrate the automotive parts for which the dividing position and/or integration has been determined among the automotive parts in the automotive body model, and
generate an optimization analysis model having a sheet thickness of the automotive part in the automotive body model as a design variable;
a sheet thickness optimization analysis condition setting unit configured to
set an objective regarding a body mass of the optimization analysis model and a constraint regarding automotive body performance of the optimization analysis model as an optimization analysis condition for performing an optimization analysis of the sheet thickness of the automotive part in the optimization analysis model, and
set a load and constraint condition given to the optimization analysis model; and
a sheet thickness optimization analysis unit configured to
perform the optimization analysis of the sheet thickness under the load and constraint condition and the optimization analysis condition set by the sheet thickness optimization analysis condition setting unit, and
obtain an optimized sheet thickness of the automotive parts in the optimization analysis model.

11. The automotive body weight reduction apparatus according to claim 10, wherein the sensitivity analysis unit is configured to
calculate an element density of each element satisfying the objective under the constraint, and
set the calculated element density of each element as sensitivity of each element.

12. The apparatus according to claim 10, wherein the automotive body model acquisition unit is configured to set all additional joining points capable of joining the parts assembly in addition to the joining points with respect to the acquired automotive body model.
